(19) United States
(12) Patent Application Publication
(10) Pub. No.: US 2004/0106821 A1
(43) Pub. Date: Jun. 3, 2004

(54) Lubricating agent containing fluorinated urethane

(76) Inventors: Ralf Grottenmuller, Burghausen (DE); Norbert Schmitt, Burgkirchen (DE); Anton Probst, Erlbach (DE)

Correspondence Address: CLARIANT CORPORATION, INTELLECTUAL PROPERTY DEPARTMENT, 4000 MONROE ROAD, CHARLOTTE, NC, 28205, US

(21) Appl. No.: 10/311,183

(22) PCT Filed: Jun. 8, 2001
(86) PCT No.: PCT/EP01/06527

(30) Foreign Application Priority Data
Jun. 15, 2000 (DE) 100 29 623.8

(51) Int. Cl. C10M 101/00; C07C 311/45; C07C 271/10

(52) U.S. Cl. 560/150; 560/158; 508/410

(57) ABSTRACT

Lubricating agent containing fluorinated urethanes Lubricants comprising fluorinated urethanes, prepared by reacting

a) compounds of the formula 1

R_{f}—X-A-H (1)

in which R_{f} denotes a perfluoroalkyl group having from 1 to 20, preferably from 4 to 16, carbon atoms, X denotes C₂-C₄ alkylene, —CON(R¹)-Q- or —SO₂N(R¹)-Q-, R¹ denotes hydrogen or C₁-C₄ alkyl, Q denotes C₂-C₄ alkylene, and A denotes —O—, —S— or —N(R¹)—, or mixtures of compounds of the formula 1 with compounds of the formula 2

R—X-A-H (2)

in which R denotes C₁-C₂₀ alkyl, preferably C₄-C₁₆ alkyl, and X and A are as defined for the formula (1),

b) triisocyanates, and

c) compounds having at least two active hydrogen atoms.

The invention relates to the use of fluorinated urethanes as ski lubricants.

Ski waxes are used in order to enhance the sliding properties of skis. Conventional ski waxes generally include relatively high molecular mass hydrocarbons such as paraffins, fatty acids, fatty acid esters, and fatty alcohols or mixtures of these and similar compounds. Certain fluorinated compounds have proven to be extremely effective ski waxes, and are employed particularly in the high-performance sport. The reason for the high effectiveness of fluorinated waxes is the coating of the ski with a fluorinated surface possessing very low surface tension, thereby greatly reducing the friction. By coating with fluorochemicals it is possible to lower the surface tension of polyethylene, for instance (31 dyn/cm) to levels of 6-18 dyn/cm. The value of 6 dyn/cm is achieved in the case of a surface composed of perfectly oriented CF₃ groups.

Some fluorinated compounds which can be used as ski lubricants are already known. For example, WO 89/10950 describes the addition of PTFE micropowder to unfluorinated ski waxes. The molar weight of the PTFE is preferably 50 000-400 000 g/mol and the particle size is less than 15 μm.

EP 0 132 879 describes the synthesis of relatively long-chain perfluoroalkanes of the formula F(CF₂)ₙF and also their use as lubricants for surfaces.

EP 0 444 752 describes the use of fluorinated diblock compounds of the general formula F(CF₂)ₙ—(CH₂)ₘH, where n=3-15 and m=5-23, as ski wax. These compounds have the advantage of compatibility with unfluorinated paraffin waxes, owing to the hydrocarbon block. DE 4 139 765 describes oligomers of fluorinated olefins of the formula F(CF₂)ᵣ—CH═CH₂. The product can be prepared by free-radical oligomerization of said olefins and is suitable as a lubricant for a variety of surfaces.

Ski waxes can be given a variety of additions in order to prevent the electrostatic charging which comes about as the result of friction and which may lead to the adherence of a water film. CH 660 018, for example, describes the use of graphite for the purpose of increasing the conductivity.

It has now been found that fluorinated urethanes can also be used as ski waxes.

The invention provides lubricants which comprise a fluorinated urethane. This fluorinated urethane is obtained by reacting

a) compounds of the formula 1

R_{f}—X-A-H (1)

in which R_{f} denotes a perfluoroalkyl group having from 1 to 20, preferably from 4 to 16, carbon atoms, X denotes C₂-C₄ alkylene, —CON(R¹)-Q- or —SO₂N(R¹)-Q-, R¹ denotes hydrogen or C₁-C₄ alkyl, Q denotes C₂-C₄ alkylene, and A denotes —O—, —S— or —N(R¹)—, or mixtures of compounds of the formula 1 with compounds of the formula 2

R—X-A-H (2)

in which R denotes C₁-C₂₀ alkyl, preferably C₄-C₁₆ alkyl, and X and A are as defined for the formula (1),

b) triisocyanates, and

c) compounds having at least two active hydrogen atoms.

In the formula 1, R_{f} can be a linear or branched perfluoroalkyl group. These groups normally contain exclusively fluorine atoms, although these perfluoroalkyl groups may also contain a certain number of hydrogen atoms or chlorine atoms. Besides compounds of the formula 1 containing perfluoroalkyl groups exclusively, mixtures with the analogous compounds of the formula 1 containing an alkyl group instead of the perfluoroalkyl group are also suitable. Preference is given to compounds of the formula 1 in which A denotes oxygen, Q and X denote ethylene, and R¹ denotes hydrogen or C₁-C₂ alkyl.

As trifunctional isocyanates it is possible, for example, to use trimerization products of aliphatic diisocyanates and trifunctional aromatic isocyanates of the following formulae:

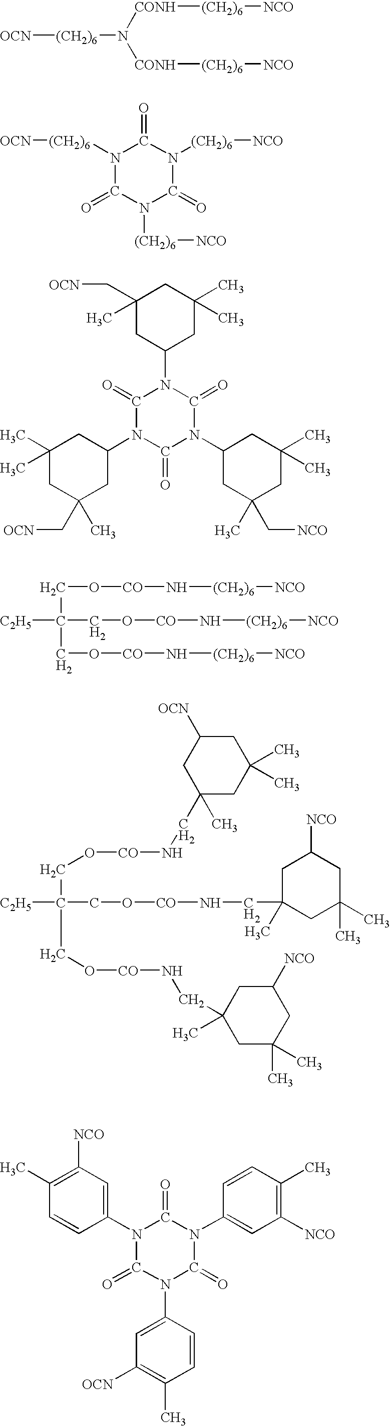

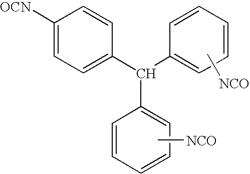

Examples of compounds having at least two active hydrogen atoms are compounds which contain two or more hydroxyl groups, thiol groups or amino groups. Specifically, the following types of compound are suitable here:

Compounds having two active hydrogen atoms:

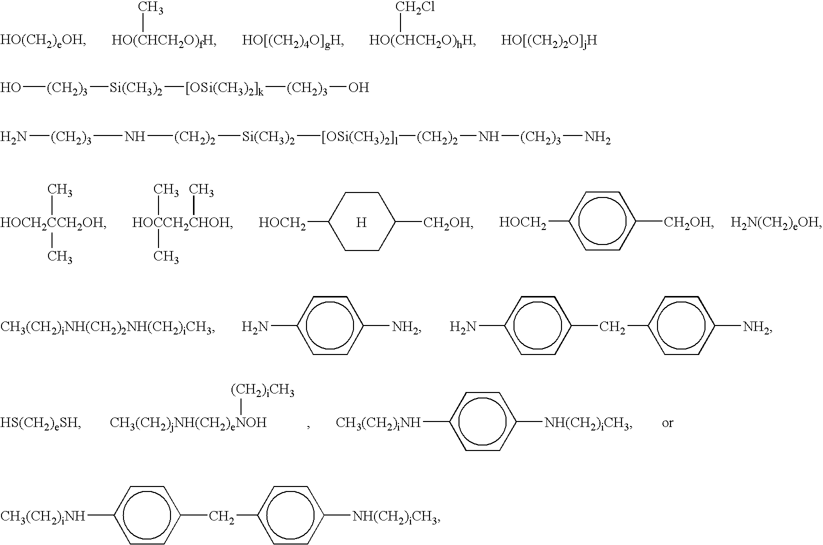

In these formulae e denotes an integer from 1 to 20, f, g, and h each denote an integer from 1 to 50, j, k, and l each denote an integer from 1 to 100, and i denotes an integer from 0 to 20.

Compounds having three active hydrogen atoms:

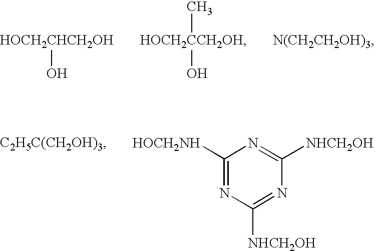

Compounds having more than three active hydrogen atoms:

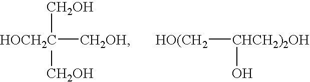

Preference is given to compounds containing two hydroxyl groups, especially compounds of the above-indicated formula

HO(CH₂)ₑOH

Depending on snow and weather conditions, the requirements imposed on the physical and chemical properties of a ski wax differ greatly. Through a suitable choice of isocyanate component and also of the nature and amount of the fluoroalcohol and of the fluorine-free alcohols and/or amines, it is readily possible to vary the physicochemical properties of the active urethane substance within wide ranges and to adapt them to the requirements. It is also possible to use combinations of two or more of the isocyanates described. Long-chain fluoroalcohols ≧C₈F₁₇ and aromatic isocyanates, for example, increase the hardness of the active substance, while shorter fluoroalcohols and suitable fluorine-free alcohols such as branched 2-ethylhexyl alcohol and unsaturated oleyl alcohol produce soft active substances which are of high viscosity at room temperature. Examples of fluoroalcohols which can be used include compounds of the structure F—(CF₂)ₙ—CH₂—CH₂—OH (®Fluowet EA—Clariant; ®Zony TAN—Dupont) or F—(CF₂)ₙ—SO₂—N(C₂H₅)—CH₂—CH₂—OH (®Fluorad FC—3M). By incorporating fluorine-free components, moreover, miscibility or compatibility with fluorine-free ski waxes can be produced. By using polyisocyanates instead of triisocyanates it is possible to raise the molar weight and hence the abrasion resistance of the wax. By using diisocyanates instead of triisocyanates, on the other hand, it is possible to lower the molar weight. Where there is only partial saturation of the triisocyanates described with monofunctional fluorinated and fluorine-free components, the remaining isocyanate groups may be crosslinked with difunctional components to give significantly higher molar weights. Examples of suitable difunctional crosslinkers include polyethylene glycol, polypropylene glycol, polytetramethylene glycol, perfluoropolyethers, α,ω)-functional polysiloxanes, and α,ω-functional hydrocarbons. The incorporation of a crosslinker component affords the possibility of influencing the hardness and the solubility as well as the molar weight. Soft films can be prepared, for example, by means of α,ω-functional polysiloxanes and perfluoropolyethers, and polysiloxanes also increase the solubility in organic solvents. Instead of the bifunctional crosslinker components, the isocyanates can also be crosslinked by adding small amounts of water.

The active urethane substances are synthesized by reacting isocyanates with the alcohol and/or amine components at elevated temperatures of 50-150° C. Where two or more different isocyanates or alcohols and/or amines are used, then all of the components may be introduced together at the start or the components can be metered in in a plurality of stages. Depending on the viscosity and hardness of the active substance, it may be necessary to add inert solvents. In the case of components which are slow to react, the reaction can be accelerated by adding known catalysts such as amines (e.g., 1,4-diaza[2.2]bicyclooctane) or organotin compounds (e.g., tin octoate). The progress of the reaction is determined by IR spectroscopy from the signal of the isocyanate group at 2275-2265 cm⁻¹.

The urethane ski waxes of the invention can be used in a variety of ways. The wax can be applied to the ski as a solution in one or more organic solvents, preferably in a concentration of 0.5-5%, or as a solid. Where the wax is to be used in the form of a solution, a suitable solvent must be chosen, in dependence on the fluorine content. At a high fluorine content, it may be necessary to use fluorinated solvents such as perfluorohexane, 1H-perfluorohexane or Frigen products, for example. At low fluorine contents, nonfluorinated solvents as well are suitable, such as ethyl acetate, butyl acetate or THF, for example. Mixtures of two or more solvents may also be used. A further possibility is to employ the active substance in the form of a suspension or dispersion.

The fluorinated active substances described are suitable not only as ski waxes but also for coating surfaces of a wide variety of kinds such as metal, plastic, and glass, for example. The coating applies a hydrophobic and oleophobic film to the surface in question, and this film greatly reduces the friction. In comparison to all fluorine-containing ski waxes known according to the current state-of the art, the urethane active substances described here have a much higher molar weight. As compared with their low molecular mass counterparts, compounds of higher molecular mass possess better abrasion resistance on the ski, so that the wax coat is durable over a greater distance traveled with the ski. The incorporation of fluorine-free alcohol and amine components into the active substance affords the advantage of miscibility with fluorine-free ski wax components and, in addition, compatibility with the material of the ski surface.

### EXAMPLES

Four examples are described which were synthesized in accordance with the following general instructions. The stated components were weighed out together into a 500 ml flask equipped with stirrer, reflux condenser, and heating bath, and were reacted with stirring at the stated temperature under an N₂ atmosphere. The completeness of the reaction was monitored by means of IR spectroscopy.

| | |
|---|---|
| Example 1: | Active substance A |
| | 266 g of 1,1,2,2-tetrahydroperfluorodecanol (® Fluowet |
| | EA, Clariant) |
| | 50 g of toluene diisocyanate |
| | 100 g of butyl acetate |
| | conditions: 125° C., 12 h |
| Example 2: | Active substance B |
| | 240 g of 1,1,2,2-tetrahydroperfluorodecanol (® Fluowet |
| | EA, Clariant) |
| | 100 g of ® Desmodur N 3300 (Bayer) |
| | 100 g of butyl acetate |
| | conditions: 125° C., 10 h |
| Example 3: | Active substance C |
| | 160 g of 1,1,2,2-tetrahydroperfluorodecanol (® Fluowet |
| | EA, Clariant) |
| | 100 g of ® Desmodur N 3300 (Bayer) |
| | 17 g of polyglycol 200 |
| | 100 g of butyl acetate |
| | conditions: 125° C., 10 h |
| Example 4: | Active substance C |
| | 120 g of 1,1,2,2-tetrahydroperfluorodecanol (® Fluowet |
| | EA, Clariant) |
| | 48 g of lauryl alcohol |
| | 150 g of ® Desmodur N 3300 (Bayer) |
| | 25 g of polyglycol 200 |
| | 100 g of butyl acetate |
| | conditions: 125° C., 10 h |

For application, the active substances were dissolved to 5% by weight in butyl acetate. The performance tests were conducted using the solutions.

Application

The tests on skis are tested by service personnel who are specialized in such tests. The skis available are prepared with a reference wax. The test track is traversed five times with each of the skis thus prepared. The best and worst times are deleted and the remainder are averaged. This gives the zero values for each pair of skis. After dewaxing, the skis are prepared with the test waxes and the same test track is traversed, again five times. The times measured were treated as described above. The resulting averages were referred to the zero values for each pair of skis, and the percentage change was determined. A negative difference denotes a shorter time for the test wax and hence better and quicker running properties.

The skis were first waxed with a paraffin/fluorine wax mixture (e.g.: Start SF 40) and then with standard fluorine waxes (e.g.: Start SF 70) and, respectively, with the fluorinated urethanes of the invention.

| Example 1: | | |
|---|---|---|
| Air temperature: | −0.6° C. | |
| Snow temperature: | −0.9° C. | |
| Air humidity: | 90% | |
| Test time: | evening | |
| Other: | wet new snow, snowfall | |
| Fluoro compound: | Compound B | Compound A |
| Difference: | −1.8% | ±0% |
| Example 2: | | |
| Air temperature: | −3.4° C. | |
| Snow temperature: | −6.9° C. | |
| Air humidity: | 90% | |
| Test time: | midday | |
| Other: | cold new snow | |
| Fluoro compound: | Compound B | |
| Difference: | −1.3% | |
| Example 3: | | |
| Air temperature: | −1.5° C. | |
| Snow temperature: | −2.0° C. | |
| Air humidity: | 88% | |
| Test time: | evening | |
| Other: | wet new snow, snowfall | |
| Fluoro compound: | Compound C | Compound D |
| Difference: | +0.5% | ±0% |

1. A lubricant comprising fluorinated urethanes, prepared by reacting
a) compounds of the formula 1
R_{f}—X-A-H (1)
in which R_{f} denotes a perfluoroalkyl group having from 1 to 20, preferably from 4 to 16, carbon atoms, X denotes C₂-C₄ alkylene, —CON(R¹)-Q- or —SO₂N(R¹)-Q-, R¹ denotes hydrogen or C₁-C₄ alkyl, Q denotes C₂-C₄ alkylene, and A denotes —O—, —S— or —N(R¹)-, or mixtures of compounds of the formula 1 with compounds of the formula 2
R—X-A-H (2)
in which R denotes C₁-C₂₀ alkyl, preferably C₄-C₁₆ alkyl, and X and A are as defined for the formula (1),
b) triisocyanates, and
c) compounds having at least two active hydrogen atoms.

2. The lubricant of claim 1, characterized in that it is a ski wax.

3. The use of the lubricant of claim 1 as a solid, solution or suspension in a suitable solvent.

4. The use of the lubricant of claim 1 as an individual component or as a mixture with one or more other kinds of fluorinated or fluorine-free components.
